(19) United States
(12) Patent Application Publication
(10) Pub. No.: US 2023/0073054 A1
(43) Pub. Date: Mar. 9, 2023

(54) MANUFACTURING METHOD OF HEMOSTATIC MATERIAL AND HEMOSTATIC MATERIAL PREPARED THEREBY

(71) Applicant: National Taiwan University, Taipei (TW)

(72) Inventors: Jiashing YU, Taipei (TW); Wei-Fan LU, Taipei (TW); Ting-Yu LU, Taipei (TW); Yi-Chen LIU, Taipei (TW)

(21) Appl. No.: 17/893,716

(22) Filed: Aug. 23, 2022

Related U.S. Application Data
(60) Provisional application No. 63/236,415, filed on Aug. 24, 2021.

(51) Int. Cl. A61L 15/28; A61L 15/42; A61L 15/32; A61L 15/56

(52) U.S. Cl. CPC A61L 15/28; A61L 15/425; A61L 15/32; A61L 15/56; A61L 2400/04

(57) ABSTRACT

A preparation method of a hemostatic material is provided, wherein the method mainly includes mixing a keratin and an alginate; obtaining a keratin-alginate composite scaffold by a freeze-gelation method; and drying the keratin-alginate composite scaffold to obtain a hemostatic material. Further, a methylene blue can be loaded into the hemostatic material so that the hemostatic material has antimicrobial photodynamic abilities.

### CROSS REFERENCE TO RELATED APPLICATION

This application claims the benefit of U.S. Provisional Application No. 63/236,415, filed on Aug. 24, 2021. The entirety of the application is incorporated herein by reference.

### BACKGROUND OF THE INVENTION

### 1. Field of the Invention

The present invention relates to a hemostatic material and a preparation method thereof, particularly to a hemostatic material comprising a keratin-alginate composite scaffold and doped with methylene blue and a preparation method thereof.

### 2. Description of Related Art

Recently, the keratin separated from human hair is widely used as a biomaterial due to its excellent biocompatibility, non-immunogenic, and biodegradability, for example, the keratin is applied in drug-delivery, tissue engineering, wound healing, and induction of cell growth and differentiation. The keratin separated from human hair is abundant and inexpensive.

The keratin separated from human hair has good liquid absorption properties, non-cytotoxicity, and biodegradability, and can enhance platelet binding and activate fibrinogen polymerization. Therefore, it is considered a promising material for wound hemostasis and repair.

### SUMMARY OF THE INVENTION

A preparation method of a hemostatic material and the hemostatic material prepared thereby are provided.

The preparation method of the hemostatic material comprises steps of: step (1): mixing a keratin solution and an alginate solution to form a mixture solution; step (2): adding a cross-linking agent solution to the mixture solution at low temperature for obtaining a keratin-alginate composite scaffold by a freeze-gelation method; and step (3): drying the keratin-alginate composite scaffold to obtain a hemostatic material.

In one embodiment of the present invention, the preparation method further comprises a step (4): doping a methylene blue into the hemostatic material.

In one embodiment of step (1), a concentration of the keratin solution is 1 to 10% (w/v); the concentration of the alginate solution is 1 to 10% (w/v), and the keratin solution and the alginate solution are mixed in a ratio of 1:1 to 10:1.

In one embodiment of step (4), the doping amount of the methylene blue per gram of the hemostatic material is 100 to 500 μg.

In one embodiment of step (1), the keratin is separated from animal hair or nails.

In one embodiment of step (2), the cross-linking agent solution is a calcium chloride solution with a concentration of 5 to 10% (w/v) using ethanol as a solvent.

In one embodiment of step (2), the freeze-gelation method is performed at a temperature below −10° C. for at least 24 hours.

The hemostatic material prepared by the above-mentioned preparation method comprises a keratin-alginate composite, wherein the keratin-alginate composite is obtained by cross-linking keratin and alginate with calcium ion as a cross-linking agent.

In one embodiment, the hemostatic material further comprises a methylene blue.

In one embodiment, a porosity of the hemostatic material is 60 to 70%.

In one embodiment, a liquid absorption capacity of the hemostatic material is 1500 to 3000%.

### BRIEF DESCRIPTION OF THE DRAWINGS

FIG. 1 shows the SEM image of the hemostatic material of an embodiment of the present invention;

FIG. 2 shows the liquid absorption of the hemostatic material of an embodiment of the present invention;

FIG. 3 shows the compression modulus of the hemostatic material of an embodiment of the present invention;

FIG. 4 shows the degradation curve of the hemostatic material of an embodiment of the present invention;

FIG. 5 shows the methylene blue release curve of the hemostatic material of an embodiment of the present invention;

FIG. 6 shows the UV-Vis absorption spectrum diagram of the hemostatic material of an embodiment of the present invention;

FIG. 7 shows the relative cell activity analysis of the hemostatic material of an embodiment of the present invention;

FIG. 8 shows the image of the colony of the culture solution of the hemostatic material of an embodiment of the present invention;

FIG. 9 shows the image of the colony of the culture solution of the hemostatic material of an embodiment of the present invention;

FIG. 10 shows the analysis diagram of the antibacterial rate of the hemostatic material of an embodiment of the present invention; and

FIG. 11 shows the image of the colony of the hemostatic material of an embodiment of the present invention.

### DETAILED DESCRIPTION OF THE PREFERRED EMBODIMENT

[Separation of the Keratin]

The keratin protein used in the present invention was separated from human hair. The hair was washed with double distilled water, air-dried, and then soaked in a chloroform/methanol (2:1, v/v) solvent for 12 hours. The solvent was evaporated to remove dirt on the hair. The hair (5 g) was then soaked in a mixed solution containing 25 mM tris(hydroxymethyl)aminomethane, 2.6 M thiourea, 5 M urea, and 5% 2-mercaptoethanol at 50° C. for three days. Next, the extraction solution was dialyzed in 1 liter of water for 36 hours (the water was changed every 12 hours) using a dialysis cassette of MWCO 1 kDa to obtain keratin.

[Preparation of the Keratin-Alginate Composite Scaffold]

In the present embodiment, the keratin-alginate composite scaffolds loaded with methylene blue were prepared by the freeze-gelation method.

The preparation step includes providing a 4% (w/v) sodium alginate solution and a 1% (w/v) keratin solution, and then mixing the sodium alginate solution and the keratin solution in a volume ratio of 1:4 at room temperature to form a keratin/alginate mixture solution; transferring the mixture solution to a plastic container; and freezing the mixture solution at −20° C. for 72 hours allowing the polymer (keratin and alginate salt) to separate from the solvent. Next, the frozen mixed solution was immersed in a pre-cooled −20° C. calcium chloride solution (8% (w/v)) using 99.5% ethanol as a solvent to induce the keratin gelation with alginate. The scaffolds were taken out of the solution and immersed in 99.5% ethanol at room temperature for 24 hours for further deposition and removal of the unreacted calcium chloride. Finally, the composite scaffold was air-dried at room temperature to obtain a hemostatic material, which was stored in a moisture-proof box for future use. Next, methylene blue was doped into the keratin-alginate composite scaffold with a doping amount of 400 μg per gram of keratin-alginate composite scaffold to obtain the hemostatic material with antimicrobial photodynamic activity.

In the following test examples, the hemostatic material without methylene blue prepared according to the above method was applied as Example 1, and the hemostatic material doped with methylene blue was applied as Example 2. The hemostatic material which is made of pure alginate was taken as Comparative Example 1.

[Evaluations of the Properties of the Keratin-Alginate Hemostatic Material]

The surfaces of the hemostatic materials of Example 1, Example 2, and Comparative Example 1 were observed by scanning electron microscopy (SEM) and were tested for porosity and liquid absorption at different times.

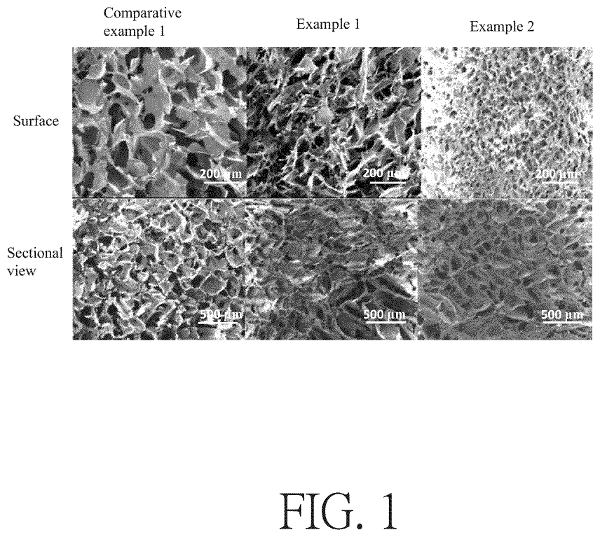

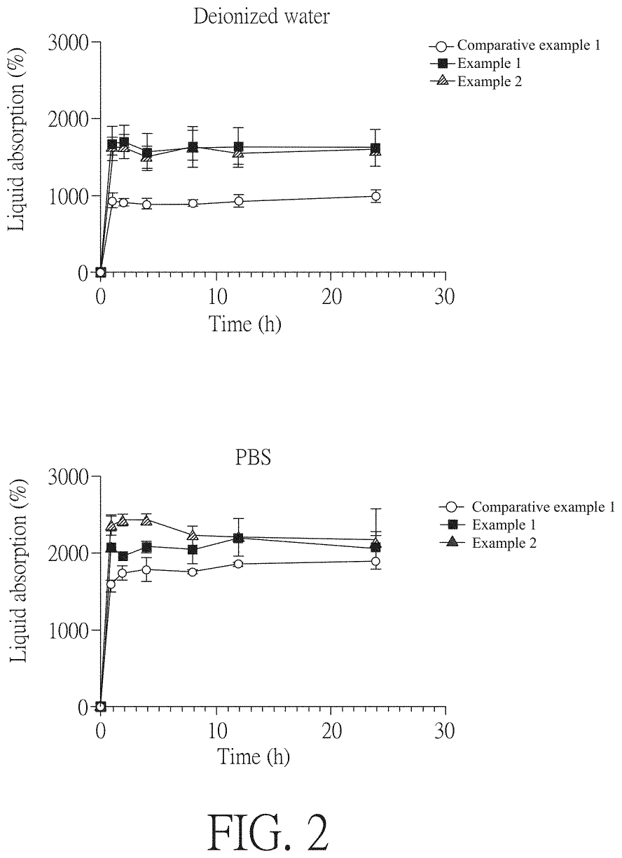

The scanning results of SEM are shown in FIG. 1, while the test results of porosity are shown in Table 1, and the test results of the liquid absorption to deionized water and phosphate-buffered saline (PBS) are shown in FIG. 2.

TABLE 1

| | | | Comparative |
|---|---|---|---|
| | Example 1 | Example 2 | example 1 |
| Porosity (%) | 67.48 ± 3.06 | 63.34 ± 1.63% | 67.35 ± 3.06 |

[Compressive Mechanical Test of the Keratin-Alginate Hemostatic Material]

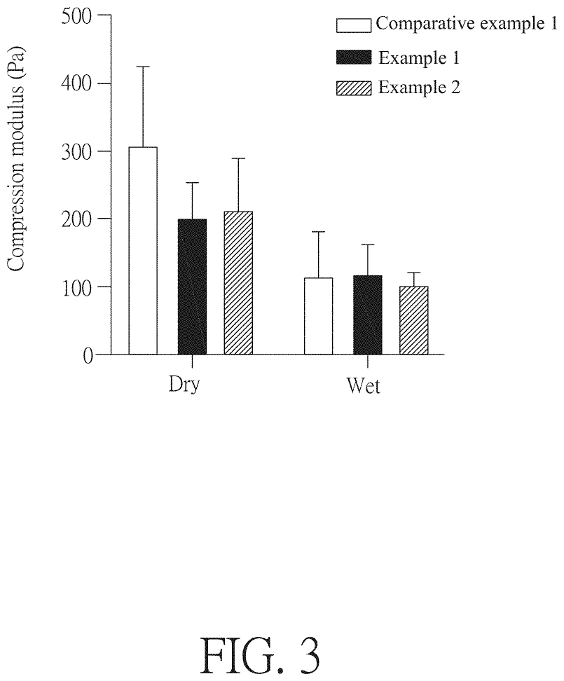

In the present compressive mechanical test, the hemostatic materials of Example 1, Example 2, and Comparative Example 1 were tested using a material testing system. To analyze the compressive strength of the three materials in the dry condition, a material with a cross-sectional area of 0.000484 m² and a height of 5.7 mm was taken and compressed to 30% of the original thickness. For the test in the wet condition, each of the materials was immersed in deionized water for 1 hour, and then analyzed at a strain rate of 10 mm/min to record the force and displacement of material compression. A stress-strain curve was established to calculate the compressive modulus. The stress-strain curve was determined by a linear fit (R²>0.98) to obtain the compressive modulus (initial linear slope) and the results are shown in FIG. 3.

Compared to the alginate hemostatic material of Comparative example 1, the compressive modulus of the hemostatic materials of Example 1 and Example 2 decreased due to the addition of keratin under dry conditions. The compressive modulus of the three hemostatic materials is not significantly different under wet conditions. Since the hemostatic materials need to be in close contact with the skin surface, the mechanical properties cannot be too rigid, and the compressive modulus of human skin is less than 35 kPa, the hemostatic materials of Example 1, Example 2, and Comparative Example 1 are all suitable for hemostasis of skin wounds.

[Degradation Analysis of the Keratin-Alginate Composite Hemostatic Materials]

In the present degradation analysis, the hemostatic materials of Example 1, Example 2, and Comparative Example 1 were immersed respectively in 20 mL of an enzyme solution in a 50 mL centrifuge tube and cultured for 2 weeks at 37° C. in a shaking incubator at 200 rpm, wherein the enzyme solution was 0.2 mg/mL trypsin dissolved in PBS. Next, at predetermined time intervals “t”, the hemostatic material was removed from the enzyme solution, thoroughly washed with deionized water, and subjected to freeze-drying to remove water. The weight (Wᵣ) of the remaining material was recorded, and each group was tested three times. The percent weight loss (%) of the material is calculated as follows:

Weight loss (%)=[(W_{dry}−Wᵣ)/W_{dry}]×100%.

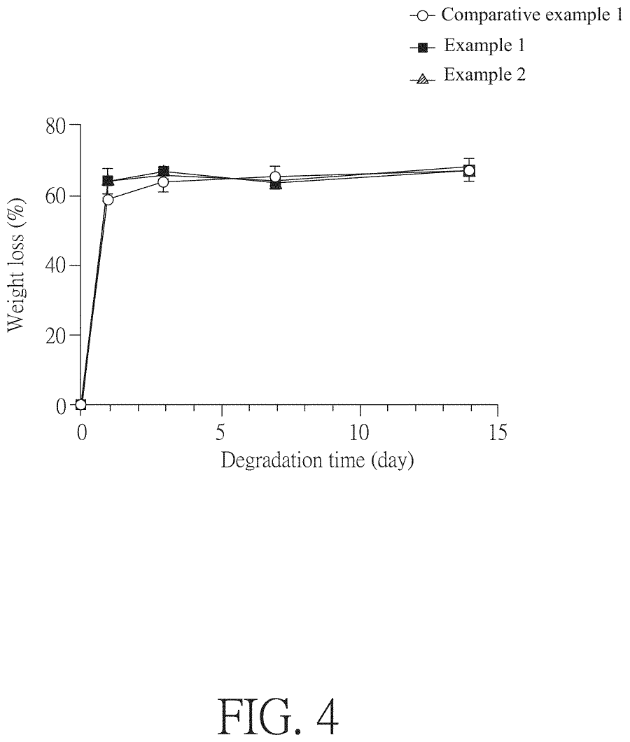

The hemostatic material needs to have proper degradation characteristics to avoid secondary damage to the wound after hemostasis. According to the results of the present degradation test, about 60% of the initial weight of the hemostatic materials of Example 1, Example 2, and Comparative Example 1 incubated in the enzyme solution for one day were lost, and its degradation curve as shown in FIG. 4. There was no significant difference in the degradation curves of the three hemostatic materials. Since the hemostatic materials of Example 1 and Example 2 did not have a chemically cross-linked structure, this may result in a larger loss of quality. Also, liquid absorption might accelerate the hydrolysis process. In addition, the results also indirectly showed that all the hemostatic materials encapsulated in the blood clot may be effectively decomposed and absorbed, and during the degradation process of the hemostatic material, the slow release of calcium ions can further help hemostasis, while the keratin in its degradation process does not cause inflammation. That is, the biodegradation rate of the keratin-alginate composite hemostatic material in the actual physiological environment is quite suitable for wound healing, and the degradation process will not cause secondary wound damage such as inflammation.

[Evaluation of the In Vitro Photosensitizer Release of the Keratin-Alginate Composite Hemostatic Material]

In this evaluation, the keratin-alginate composite hemostatic material doped with methylene blue in Example 2 was immersed in a 20 mL PBS solution stored in a 50 mL centrifuge tube, and incubated at 37° C. in a shaking incubator at 200 rpm. After the liquid samples in the centrifuge tube were collected at a predetermined time point, fresh PBS was added immediately. The content of methylene blue in the collected liquid samples was measured by UV-Vis spectrophotometer for making the calibration curve of methylene blue. This experiment was carried out at least three times.

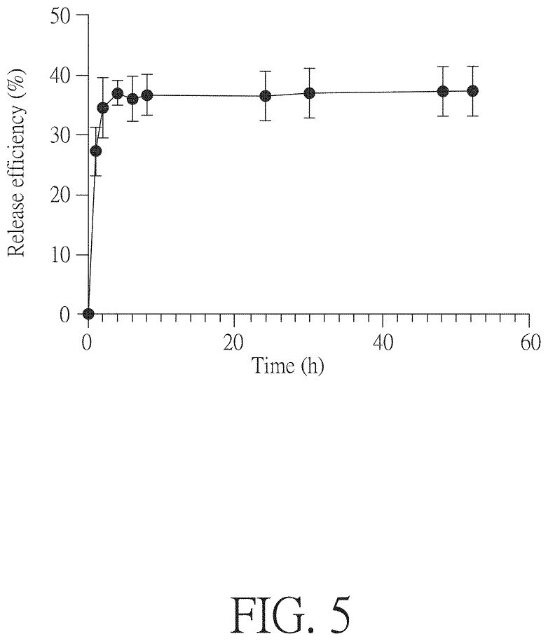

The methylene blue release curve of the scaffold of Example 2 is shown in FIG. 5, and the loading capacity and the encapsulation efficiency of methylene blue were respectively 0.03092±0.001256% (309.2±12.6 μg methylene blue/g material) and 77.30±3.14%. And as shown in FIG. 5, a rapid drug release was observed during the first hour, approximately 27.25±3.99% of methylene blue was released, followed by a sustained slow release during the next 52 hours with a cumulative release efficiency of 37.62±4.18%. It should be noted that the composite hemostatic material of Example 2 can achieve a high release rate of methylene blue by absorbing wound exudate in the early stage of wound healing, to provide antibacterial function and prevent infection.

[Evaluation of Reactive Oxygen Species Detection of the Keratin-Alginate Composite Hemostatic Materials]

In this evaluation, 0.1 g of the composite hemostatic materials of Example 1, Example 2, and Comparative example 1 were immersed in 15 mL of reaction solution in a 6-well plate. The reaction solution includes 0.025 mM N,N-dimethyl-4-nitrosoaniline (RNO) and 0.25 mM imidazole. The hemostatic materials were irritated with 650 mW/cm² of 660 nm laser light at a distance of 8.5 cm above the sample for 30 min. The solution was then diluted with 1.5 mL of water and the absorbance at a wavelength of 440 nm was measured by a UV-Vis spectrophotometer. If singlet oxygen reacts with imidazole to form imidazole endoperoxide, it will lead to RNO bleaching, and RNO has an obvious absorption peak at 440 nm, but if RNO is bleached, the peak will be reduced.

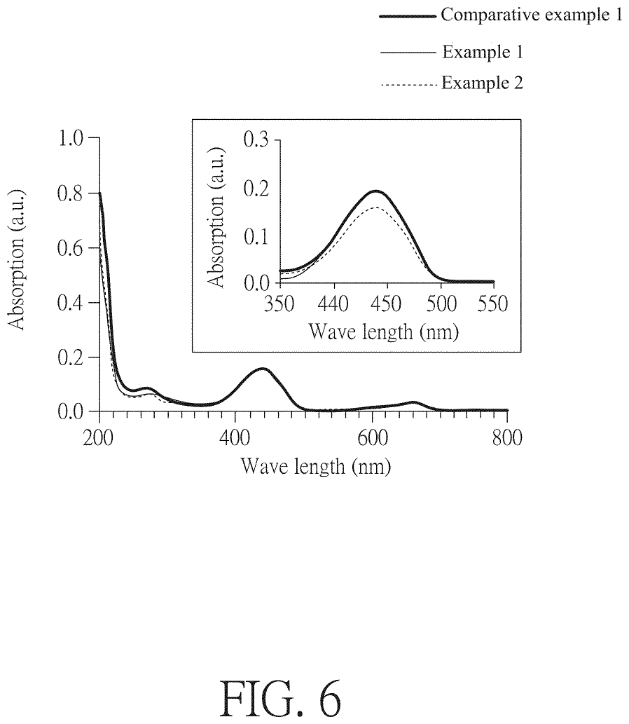

The evaluation results are shown in FIG. 6. After being irradiated by 660 nm laser light, the absorbance of the composite hemostatic material of Example 2 at 440 nm decreased, which indicates that the composite hemostatic material of Example 2 can induce the generation of the reactive oxygen species (ROS), thus becoming a potential antimicrobial photodynamic hemostatic material.

[Evaluation of Biocompatibility of the Keratin-Alginate Composite Hemostatic Material]

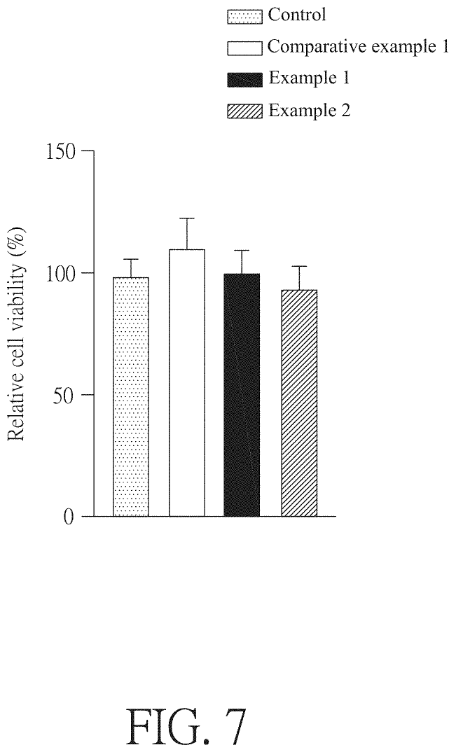

The biocompatibility test was based on the methods specified in ISO 10993-5 and ISO 10993-12. The hemostatic materials of Example 1, Example 2, and Comparative Example 1 were sterilized with ultraviolet light for 24 hours and incubated in a DMEM-HG medium at 37±1° C. for 24±2 h, respectively. The cultured medium was called extraction medium, and the culture medium without the material was cultured under the same culture conditions as a control group. NIH3T3 cells at passage 19 were seeded in 96-well plates at a density of 7,500 cells per well, and cultured overnight. After removing the medium and washing with PBS, 200 μL of the extraction medium was used to treat the cells in the 96-well plates and incubated for 24 hours. After incubation, 200 μL of MTT solution (5 mg/mL) was added to the wells and incubated for another 4 hours at 37° C., MTT solution was then removed and 200 μL of dimethyl sulfoxide (DMSO) was added to dissolve the formazan crystals and use an orbital shaker for 30 minutes to thoroughly mix the mixture. Finally, the optical density was measured by an ELISA plate reader at a wavelength of 570 nm, and the results were recorded and statistically analyzed by comparison with controls. The results are shown in FIG. 7. The test results showed that the hemostatic materials of Example 1 and Comparative Example 1 without methylene blue did not have any toxicity to the cells. However, the hemostatic material of Example 2 doped with methylene blue showed lower cell viability. The cytotoxicity was still acceptable, and the cell viability of the hemostatic materials of Example 1, Example 2, and Comparative Example 1 all exceeded 90%, which indicates that all hemostatic materials were biocompatible.

[Evaluation of the In Vitro Antimicrobial Photoinactivation Assay of the Keratin-Alginate Composite Hemostatic Material]

In the present evaluation, Gram-positive Staphylococcus aureus (S. aureus) and Gram-negative Escherichia coli (E. coli) were cultured in LB medium under aerobic conditions at 37° C. in a shaking incubator at 200 rpm for 24 hours. Next, a bacterial suspension was obtained by diluting the bacterial cultures with deionized water to a density of approximately 10⁵ CFU/mL. The hemostatic materials of Example 1, Example 2, and Comparative Example 1 were sterilized by ultraviolet radiation for 30 minutes, and then transferred to a 24-well plate containing a bacterial suspension, about 10 mg of the hemostatic material and 1 mL of bacterial suspension were cultured together. The hemostatic materials were then incubated in the dark or irradiated with 660 nm laser light at an intensity of 650 mW/cm² for 30 minutes, the laser lamp was placed at a distance of 8.5 cm above the sample to avoid overheating. Afterward, 100 μL of treated bacterial suspension (undiluted and serially diluted to ˜10⁴ or ˜10³ CFU/mL), and untreated bacterial suspension as a control group were evenly inoculated on LB (Lysogeny broth) agar and incubated at 37° C. for 24 hours. The number of colonies was then counted, and the relative antibacterial rate of the hemostatic material was evaluated according to the number of colonies. The calculation of the relative antibacterial rate is shown in the following formula:

Relative antibacterial rate (%)=(N_{Control}−N_{Sample})N_{Control}×100%

In the above formula, N_{Control} is the average number of colonies in the dark group, and N_{Sample} is the number of colonies in the sample group. The experiment was performed three times. The bacterial suspension with a density of about 10⁶ CFU/mL was obtained by culturing the samples overnight in an LB medium. Then, the bacterial suspension was inoculated onto LB agar using a sterile swab, and the hemostasis materials of Example 1 and Example 2 were placed on LB agar and incubated in the dark or irradiated with 660 nm laser light at an intensity of 650 mW/cm² for 30 minutes. After overnight incubation at 37° C., hemostatic materials were removed and the bacterial growth was controlled.

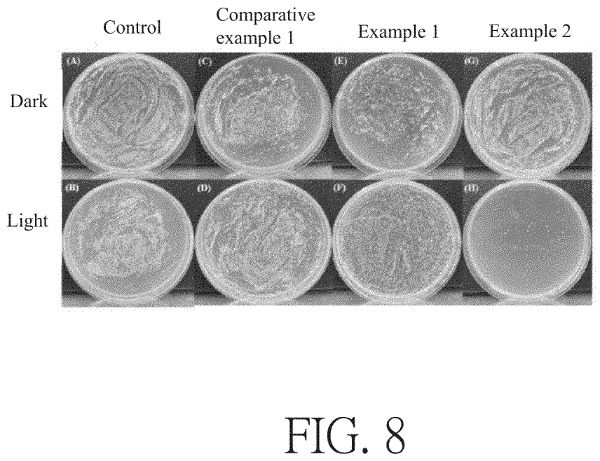

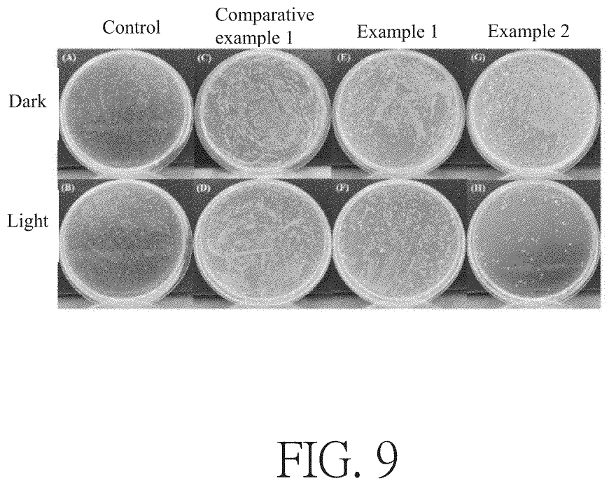

The relative antibacterial rate was directly calculated from the number of viable bacterial colonies on LB agar. The images of colonies irradiated by laser light and cultured in the dark are shown in FIG. 8 (S. aureus) and FIG. 9 (E. coli).

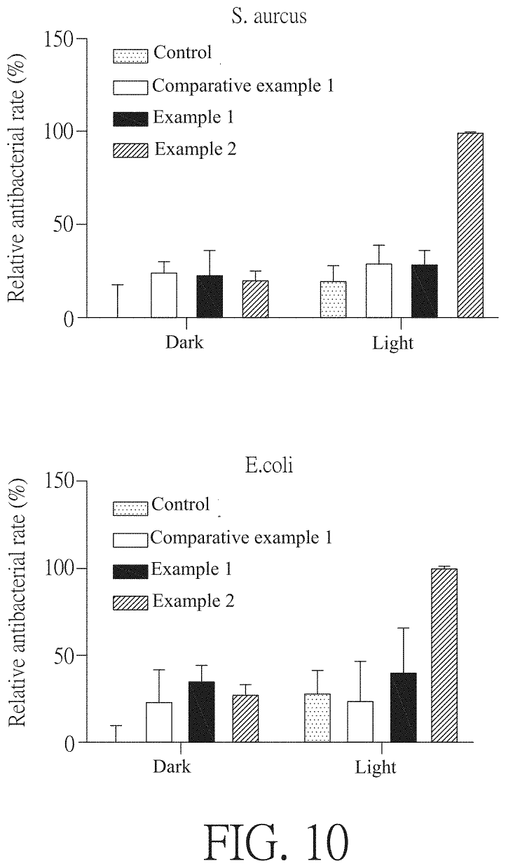

According to FIG. 8 and FIG. 9, it should be noted that there were many colonies after being irradiated with laser light or cultured in the dark for 30 minutes, which means that the laser light irradiation has no effect on bacterial viability, and all groups of hemostatic materials were cultured in the dark neither showed an obvious antibacterial effect. Please refer to the antibacterial rate analysis in FIG. 10, the hemostatic material without methylene blue in Example 1 still did not show its antibacterial ability after being irradiated with laser light. However, the hemostatic material doped with methylene blue in Example 2 showed excellent antibacterial ability after being exposed to light at 660 nm for 30 minutes. The relative antibacterial rates for Staphylococcus aureus and Escherichia coli were 99.95±0.05% and 99.68±0.55% respectively, which showed that the antimicrobial photodynamic can be triggered by irradiation.

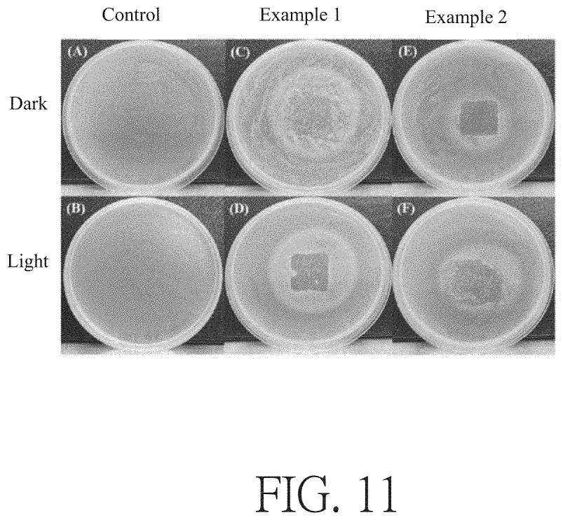

The antibacterial rate of the attached hemostatic material was to simulate the antibacterial situation when the hemostatic material is used as a hemostatic patch. The results are shown in FIG. 11, wherein the hemostatic material of Example 1 had a significant inhibitory effect on the growth of Escherichia coli in the dark or in light. When the hemostatic material of Example 2 was cultivated in the dark, its antibacterial effect could not be determined; however, the growth of the colonies inoculated on LB agar was inhibited under irradiation.

Based on the results of the above evaluations, it can be understood that the hemostatic material provided by the present invention prepared by the freeze-gelation method has a high liquid absorption rate and excellent biocompatibility, and is biodegradable. When the hemostatic material is doped with methylene blue, it has an antimicrobial photodynamic effect. The methylene blue will be released and provide an antibacterial effect after being illuminated. Therefore, when the hemostatic material of the present invention is attached to the bleeding wound, it can absorb a large amount of blood and exudate. The hemostatic material also provides an antimicrobial photodynamic function to achieve hemostasis and antibacterial effects, and its biodegradable properties can avoid secondary damage to the wound when the hemostatic material is removed from the wound.

1. A preparation method of a hemostatic material, comprising steps of:
step (1): mixing a keratin solution and an alginate solution to form a mixture solution;
step (2): adding a cross-linking agent solution to the mixture solution at low temperature for obtaining a keratin-alginate composite scaffold by a freeze-gelation method; and
step (3): drying the keratin-alginate composite scaffold to obtain a hemostatic material.

2. The preparation method claimed in claim 1, further comprising a step (4): doping a methylene blue into the hemostatic material.

3. The preparation method claimed in claim 1, wherein in step (1), a concentration of the keratin solution is 1 to 10% (w/v); the concentration of the alginate solution is 1 to 10% (w/v), and the keratin solution and the alginate solution are mixed in a ratio of 1:1 to 10:1.

4. The preparation method claimed in claim 2, wherein a doping amount of the methylene blue per gram of the hemostatic material is 100 to 500 μg.

5. The preparation method claimed in claim 1, wherein in step (1), the keratin is separated from animal hair or nails.

6. The preparation method claimed in claim 1, wherein in step (2), the cross-linking agent solution is a calcium chloride solution with a concentration of 5 to 10% (w/v) using ethanol as a solvent.

7. The preparation method claimed in claim 1, wherein in step (2), the freeze-gelation method is performed at a temperature below −10° C. for at least 24 hours.

8. A hemostatic material, which is prepared by the preparation method claimed in claim 1, comprising:
a keratin-alginate composite;
wherein the keratin-alginate composite is obtained by cross-linking a keratin and an alginate with calcium ion as a cross-linking agent.

9. The hemostatic material claimed in claim 8, further comprising a methylene blue.

10. The hemostatic material claimed in claim 8, wherein a porosity of the hemostatic material is 60 to 70%.

11. The hemostatic material claimed in claim 8, wherein a liquid absorption capacity of the hemostatic material is 1500 to 3000%.
